(19) United States
(12) Patent Application Publication
(10) Pub. No.: US 2017/0196193 A1
(43) Pub. Date: Jul. 13, 2017

(54) SYSTEM, METHOD AND FORMULATION FOR TREATING USED ANIMAL BEDDING

(71) Applicant: GREENSCENE AGRITEK INC., Chilliwack (CA)

(72) Inventors: Paul CROSS, Chilliwack (CA); Jennine STROMKINS, Chilliwack (CA)

(73) Assignee: GREENSCENE AGRITECH INC., Chilliwack (CA)

(21) Appl. No.: 14/994,015

(22) Filed: Jan. 12, 2016

(51) Int. Cl. A01K 1/015; A61K 36/54; A61K 36/61; A61K 36/15

(52) U.S. Cl. CPC A01K 1/0155; A61K 36/15; A61K 36/54; A61K 36/61

(57) ABSTRACT

A method, system and formulation for treating animal bedding and animals is provided. For the method, it comprises: mixing used bedding with unused bedding to produce mixed bedding; deodorizing the mixed bedding; drying the mixed bedding to have a moisture content of between approximately 7 and 14%; removing lighter fibers from the mixed bedding; adding an oil treatment to the mixed bedding to have a moisture content of between approximately 7 and 14% in the mixed bedding; and bagging the mixed bedding while the mixed bedding is still moist from the oil treatment.

### TECHNICAL FIELD

This disclosure relates to a system, method and formulation for treating used animal bedding and producing treated bedding with additives, such as oil extracts.

### BACKGROUND OF DISCLOSURE

Current practices for equestrian facilities and farms is to remove daily soiled bedding from a horse's stall and transport it to a storage pile. Replacement bedding is then added to the stall and raked to provide even sleeping/resident bedding for the animal. The soiled bedding is mostly wood fiber with fecal matter, hay and grass (organics) and is stockpiled over a period of time, depending on the number of horses at the facility until the volume reaches such levels that it requires removing from the farm. Bins or other devices are then utilized to hold the used bedding, and then it is trucked away to a site or collection area, where it is dumped. There are limited uses for this used bedding at this stage, and it often becomes a nuisance material, accumulating and causing foul odours, greenhouse gas release and other environmental concerns. There have been concerns regarding horse manure build-up with contaminates entering land, air and water, causing numerous health concerns to humans.

Current used bedding disposal methods include composting, spreading on land, legal and illegal landfill dumping and stockpiling. Composting reuses the used bedding for top soil, mulch and potting soil. Spreading on land has been implemented on some crops. Studies of the long-term ramifications of this practice indicate possible nutritional imbalance and dilution of soils by the addition of wood products to the soil and water contamination caused by leaching. Landfill dumping is used when other options are not available. Transportation and landfill tipping costs are increasing significantly. Many landfills are not accepting horse manure, as this interferes with the anaerobic decomposition if not applied properly. Stockpiling is used as a cost savings method when a farm cannot afford storage bins for pickup and does not produce enough manure to make pickup attractive to the haulers. This method may have adverse effects on aquifers and properties because of odour, bacteria parasites, and leaching concerns.

In the used bedding, waste organic material therein may contain diseases or pathogens that were deposited into the bedding by feces, urine or expectorant from the horses. Also, mold spores may be in “clean” unused bedding that is subsequently provided into an animal's stall after soiled bedding. Although not usually evident, if the sawdust/wood shavings have been stored outside or are damp, these mold spores typically begins to grow within the pile or in the stalls. Horses like to eat with their heads down and may easily inhale these microscopic mold spores, potentially causing respiratory problems.

U.S. Pat. No. 5,361,708 discloses an apparatus and method for pasteurizing and drying sludge wherein the material to be dried passes through three separate cylinders while being in contact with hot gasses.

U.S. Pat. No. 5,689,941 discloses a high-density combination dry hay and hay silage livestock feed-making apparatus processing crude silage and dried powder into a consumable product which is then packaged.

There is a need for clean animal bedding material that utilizes used bedding.

### SUMMARY OF THE DISCLOSURE

A first aspect of disclosure relates to a method of processing animal bedding material. The method comprises: mixing used bedding with unused bedding to produce mixed bedding; deodorizing the mixed bedding; drying the mixed bedding to have a moisture content of between approximately 8 and 14%; removing lighter fibers from the mixed bedding; adding an oil treatment to the mixed bedding to have a moisture content of between approximately 8 and 14% (in one case approximately 10%) in the mixed bedding; and bagging the mixed bedding while the mixed bedding is still moist from the oil treatment.

The method may further comprise deodorizing the mixed bedding with bicarbonate soda after the used bedding is mixed with the unused bedding (before drying).

For the method, the oil treatment may be added through an auger on an incline that mixes the mixed bedding with the oil treatment.

In the method, the oil treatment may comprise a formulation of essential oils comprising between approximately 9 and 14% of black spruce essential oil.

In the method, the formulation of essential oils may further comprise between approximately 0.5 and 2% of cassia.

In the method, the formulation of essential oils may further comprise: between approximately 12 and 18% of Canadian balsam essential oil; between approximately 9 and 11% of douglas fir essential oil; and between approximately 22 and 26% of Canadian spruce essential oil. The formulation may further comprise: between approximately 25 and 37% of silver fir essential oil; between approximately 0.25 and 6% of litsea cubeba essential oil; between approximately 1 and 5% of peppermint essential oil; and between approximately 0.5 and 2% of cassia essential oil.

In the method, the oil treatment may further comprise a carrier lotion that comprises: between approximately 15 and 20% of cetyl alcohol (e.g. approximately between 85 and 105 mL); between approximately 40 and 60% of stearic acid (e.g. approximately between 225 and 330 mL); between approximately 10 and 20% of caprylic acid (e.g. approximately between 35 and 75 mL); between approximately 1 and 5% of polysorbate 20 (e.g. approximately between 3 and 15 mL); and between approximately 3 and 7% of Borax (e.g. approximately between 9 and 25 mL).

In the method, a composition of the oil treatment may comprise a ratio of the essential oils to the carrier lotion to water as being approximately expressed as essential oils 1 : carrier lotion 0.933 : water 0.0765.

In the method, approximately 18.9 L of the oil treatment may be mixed with approximately 80 bales (approximately 240 ft³) of the mixed bedding.

In a second aspect, an oil treatment for treating animal bedding material is provided. The oil treatment comprises a formulation of essential oils comprising: between approximately 9 and 14% of black spruce essential oil; and between approximately 0.5 and 2% of cassia.

In the oil treatment, the formulation of essential oils may further comprise: between approximately 12 and 18% of Canadian balsam essential oil; between approximately 9 and 11% of douglas fir essential oil; and between approximately 22 and 26% of Canadian spruce essential oil.

In a third aspect, another oil treatment for treating animal bedding material is provided. The oil treatment comprises a formulation of essential oils comprising: between approximately 0.25 and 6% of manuka essential oil; and between approximately 0.05 and 4% of litsea cubeba essential oil.

The oil treatments of the second and third aspects may further comprise a carrier lotion that comprises: between approximately 15 and 20% of cetyl alcohol; between approximately 40 and 60% of stearic acid; between approximately 10 and 20% of caprylic acid; between approximately 1 and 5% of polysorbate 20; and between approximately 3 and 7% of Borax.

For the oil treatments, they may comprise a ratio (in mL) of the essential oils to the carrier lotion to water as approximately essential oils 250: carrier lotion 3750 : water 16000.

For the oil treatments, wherein approximately 18.9 L of an oil treatment may be mixed with approximately 240 ft³ of the mixed bedding.

### BRIEF DESCRIPTION OF THE DRAWINGS

Embodiments of the present disclosure will now be described, by way of example only, with reference to the accompanying drawings, in which:

FIG. 1 is a flow chart of a process for treating used animal bedding that in part treats processed bedding with an oil formulation according to an embodiment;

FIG. 2 is a block diagram of treatment processing modules of an exemplary industrial process carrying out processes as shown in FIG. 1, including an oil treatment module to process bedding with an oil formulation according to an embodiment; and

FIG. 3 is a block diagram of components of the oil treatment module as shown in

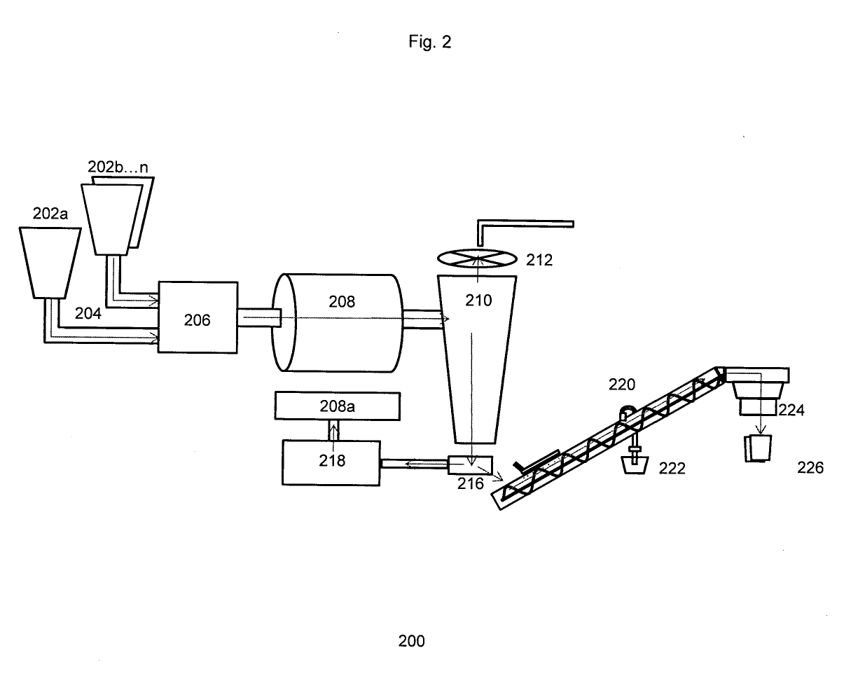

FIG. 2, according to an embodiment.

### DETAILED DESCRIPTION OF EMBODIMENTS

The description which follows and the embodiments described therein are provided by way of illustration of an example or examples of particular embodiments of the principles of the present disclosure. These examples are provided for the purposes of explanation and not limitation of those principles and of the present disclosure. In the description which follows, like parts are marked throughout the specification and the drawings with the same respective reference numerals.

Briefly, an embodiment processes used animal bedding materials, such as used horse bedding collected from stalls in stables, combines the used bedding with additional bedding materials (which may include unused, clean bedding material) to produce mixed bedding materials and then treats the mixed bedding materials to produce a new, usable bedding product. Clean bedding generally comprises mostly of wood products such as a combination of wood shavings, woodchips, sawdust, waste hay and other fibrous materials. Used bedding typically comprises approximately 60% to 80% wood product, 6% waste hay that is dropped by the animal while eating, and 15% to 30% animal feces. Urine makes animal bedding between 30% and 50% wet. Animal bedding may be for any type of animal that is housed in a farm (commercial or hobby), including as horse and dogs, etc.

As part of a process of an embodiment, the bedding material may be exposed to one or more of heat and ultraviolet (UV) light exposure to kill microbes and/or spores that were present in the used bedding (or even the new bedding). Heating the mixed bedding heats ammonia in waste organic material in the used bedding to vaporize nitrogen and hydrogen in the ammonia, removing it to reduce and/or eliminate the related odours. Heating the bedding material also dries feces present in the used bedding into a fine powder that can be removed in a dust processing system, to reduce or eliminate odours and pathogens originating from the fecal material. Finally, the bedding material may be further treated with a blend of therapeutic treatments (such as plant oils) to for one or more treatments, such as insect repellant, antibacterial properties and odour suppression.

The term “used bedding material” or “used bedding” herein refers to material provided as input to the bedding processing system of an embodiment. Used bedding may include, but is not limited to, any one or more of waste bedding, waste material, manure, sawdust, particulate, other loose materials, pellets, straws, paper, hemp/flax, peat moss, woodchips, grains, minerals, seeds, liquefied manure, slurries, liquids and liquids containing waste material. Wet used bedding, on average contains approximately between 30% and 50% moisture, mostly in the form of urine (which itself may be approximately 95% water). The actual moisture content depends on many factors, including on how the bedding was stored and whether used bedding piles were covered or not. It will be appreciated that an embodiment can process any transportable material, having any of waste and/or organic material therein or not.

The term unused bedding material refers to bedding that is not used or clean and/or has not yet been processed as recycled bedding. The term “mixed bedding material” or “mixed bedding” herein refers to bedding materials that are being processed by an embodiment and may be a mixture of used and unused bedding material. The term recycled bedding refers to mixed bedding that has been treated by a process of an embodiment (or another process) and is the output of the process.

Embodiments described herein may be adapted to process a wide variety of organic products to dry and/or decontaminate them.

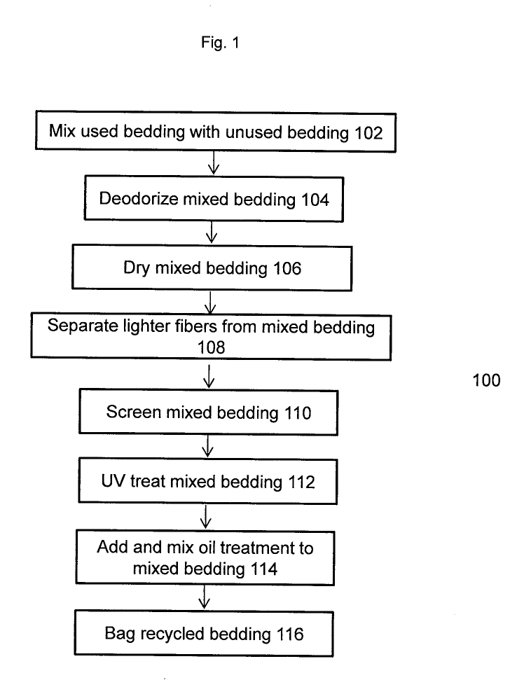

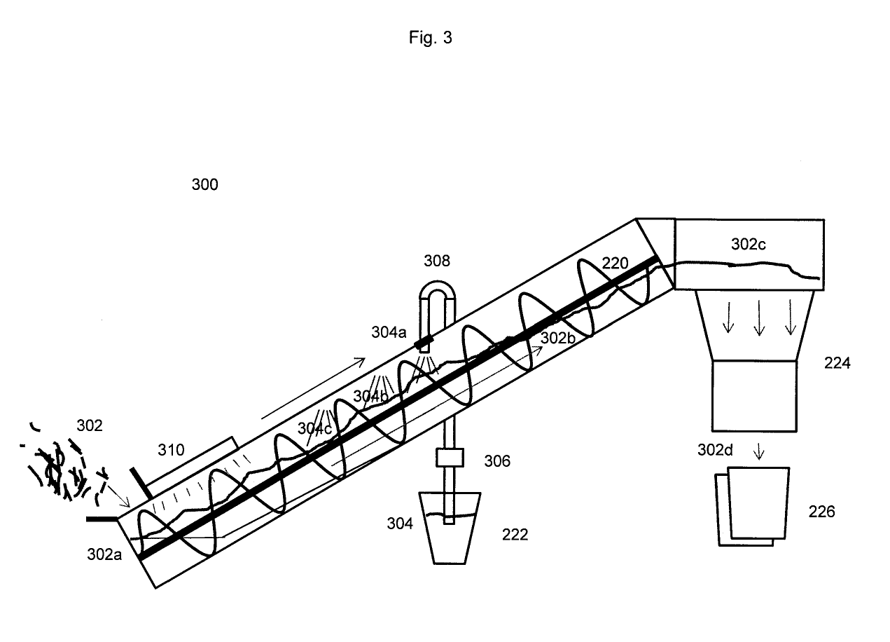

With reference to FIGS. 1-3 details are provided on exemplary processes, systems and formulations for embodiments to produce a bedding product from a mixture of used bedding and new bedding. The exemplary processes comprise in part animal bedding process 100 (FIG. 1), bedding processing system 200 (FIG. 2) showing components implementing features of process 100 and oil treatment module 300 (FIG. 3) providing more details on components implementing oil treatment process 114 (FIG. 1) of process 100.

In FIG. 1, process 100 starts with mixing used bedding with unused bedding in process 102 to provide the input material for process 100. In FIG. 2, hoppers 202a and 202b. . . n are provided that separately receive different types of bedding (e.g. used bedding having a first moisture content, used bedding having a second moisture content, unused bedding having a third moisture content, unused bedding having a fourth moisture content, used overflow materials recycled from process 100 etc.) and each type of bedding is conveyed on conveyors 204 to mixer 206 which collects the types of beddings from hoppers 202 and mixes them together via a variable frequency drive controlled auger/blade (not shown) that can control the rate of insertion of bedding from each hopper as needed to form a relatively well-mixed aggregation of bedding materials. In one embodiment, the input material comprises approximately: 15-20% new bedding, 20-40% previously processed bedding, and 40-65% used bedding. Mixing may be conducted for approximately 15 minutes to ensure thorough blending of bedding materials from hoppers 202.

As part of the mixing process, the mixed bedding may be deodorized per process 104 (FIG. 1). This may involve adding bicarbonate soda to the mixed bedding through a sieve. The amount of bicarbonate soda added is approximately ¼ pounds (0.1136 kg) of bicarbonate soda to the equivalent of one 32 lb. (14.55 kg) bag of mixed bedding. This mixing rate is provided using a variable frequency drive, where a 50 lb. bag of bicarbonate soda is metered to be added to the mixed bedding at a rate of 10 lb. per hour. Converted to metric units, the amount of bicarbonate soda added to the bedding is approximately 0.89 kg of bicarbonate soda per m³ of bedding. Typically, 10 lbs. of bicarbonate soda is provided for approximately 180 cubic feet of mixed bedding (depending on moisture content). Alternatively, some or all of the bicarbonate soda may be added at a later process if sufficient water is provided to activate the bicarbonate soda.

Next, the mixed bedding is dried per process 106 (FIG. 1). To dry the mixed bedding, the mixed bedding is conveyed from mixer 206 (FIG. 2) to dryer 208, which has heater 208a (which itself may be a multi-staged drying system). Heater 208a may generate heat from burning multiple fuel sources, including burning of bedding “fines” as extracted from the system. Dryer 208 may have an auger/blade (not shown) to further agitate the mixed bedding to expose the contents to heat in dryer 208.

An initial processing temperature in dryer 208 may be between approximately 400 F (approx. 204° C.) and 1000 F (approx. 538° C.). Exposure to this temperature causes water in the urine in the used bedding of the mixed bedding to be turned to steam, which has an effect of sterilizing the mixed bedding. The heated air temperature in dryer 208 preferably is made to be high enough to kill pathogens present in the mixed bedding, having regard for the time that the mixed bedding will be present in dryer 208, but low enough to not char the cellulose and wood fiber material present in the mixed bedding. In one configuration, the mixed bedding may be in dryer 208 for approximately 3 minutes or less at the above noted temperature range.

Dryer 208 may have internal heaters and agitators to reduce an average moisture content in the mixed bedding to be any desired value. For one embodiment a target moisture level for in the mixed bedding leaving dryer 208 is to be approximately less than 10% moisture. In one embodiment a moisture content of between about 8% and 12% is preferred. In another embodiment a moisture content of between about 7% and 14% is preferred. When the moisture content is around approximately 7%, it has just sufficient moisture to allow pelletization of the fibers, if that is desired. If moisture level in the mixed bedding is too low, some moisture may be re-added to the mixed bedding to bring the moisture content to an acceptable level. If the moisture level of the bedding added to dryer 208 is too low, that bedding may be ignited during the drying process. Bedding having a moisture content of about 6% and less is more susceptible to ignition.

In another embodiment for dryer 208, the temperature of the mixed bedding leaving dryer 208 is approximately at least 150 F (66° C.), but preferably around approximately 190 F (88° C.).

Next, the now dried and deodorized mixed bedding (which contains used bedding and unused bedding) is processed to separate the lighter fibers (which are too small to be kept in the final mixed bedding) in separation process 108 (FIG. 1). The finer particles may contain fecal dust (remaining from the heating in dryer 208 and fine organics, such as wood dust, grass and hay. To separate the lighter fibers from the heavier fibers in the mixed bedding, the mixed bedding is conveyed from dryer 208 (FIG. 2) to cyclone 210, which has a suction system 212 to draw up dust and very fine particles from the mixed bedding. The drawn up dust, fine particles and air leave the top of cyclone 210 and the remaining heavier particles fall to the bottom of cyclone 210.

Next, the particles of the mixed bedding exiting the bottom of cyclone 210 are screened in process 110 (FIG. 1) to remove the small and medium sized particles that are not large enough to be used in the mixed bedding. In screening the mixed bedding, the flow of mixed bedding from the bottom of cyclone 210 (FIG. 2) is fed through screen 216, which agitates the mixed bedding to remove the smaller sized particles for collection in collection system 218. These smaller sized particles may be pelletized, used as fuel for heater 208a, used as a soil amender, fertilizer or top soil additive.

Next, the mixed bedding that has not been collected and passes through screen 216 is destined for further treatment and processing by adding an oil treatment to it in process 114. This mixed bedding is fed to auger 220 where an oil treatment is dispensed into auger 220 to mix be mixed with the mixed bedding. In one embodiment, auger 220 has a variable frequency drive to permit different conveying (and therefor mixing) speeds to be imparted on the mixed bedding as it moves upward through auger 220 from its entry point to a higher exit point in auger 220. As the mixed bedding is churned an moved upward in auger 220, the oil treatment is blended into the mixed bedding.

When the mixed bedding leaves auger 220, the mixed bedding is processed by bagging system 224 for bagging a fixed amount of bedding into bags 226. Typical industry bags hold 32 lbs. Bags 226 are sealed so that the mixed bedding is still moist with a moisture content reflecting the mixed bedding being infused with the oil treatment. In one embodiment relative level of moisture in the bagged bedding that has been infused with the oil treatment is between approximately 9 and 12%. In another embodiment the moisture level is between about 7% and 14%. When the bag is opened and the contents spread for use in an animal's stall, the moisture level retained in the bagged bedding allows aromatics contained bedding and air in the bag to be released to the ambient air with the oil treatments, which may then be inhaled by the animal in the stall, facilitating the conveyance of the oil treatments to the animal's respiratory tract. Oils soaked into the mixed bedding stay in the mixed bedding allowing animals to contact the oils when they touch the mixed bedding in the stalls.

System 300 in FIG. 3 provides additional details on auger 220 (FIG. 2) and mixing system 222 for the oil treatment process 114 (FIG. 1). Auger 220 is positioned on an incline with the mixed bedding inserted at a bottom end of auger 220 with the oil treatment added into auger 220, which then mixes the mixed bedding with the oil treatment. Mixed bedding 302 that has passed through screen 216 (FIG. 2) is fed to the bottom of auger 220, where it initially collected at the bottom of auger 220 (shown as bedding 302a ). The incline may be between a 10% and 40% grade. In other embodiments, no incline is provided. Mixing system 222 has a mixing bucket and pump 306 that contains a pre-mixed amount of oil treatment that is fed through pipe 308 through an opening up a length of auger 220, to be released into auger 220 (shown entering auger 220 as oil mixture 304a and collecting downstream in auger 220 as mixtures 304b and 304c). The spray insertion of oil mixture 304a and the churn of the mixed bedding provided by auger 220 allows for a good mixing of oil mixture 304 in bedding 302. If the insertion rate of the oil mixture exceeds the upward travel rate of auger 220, some oil mixture may collects at the bottom end of auger 220. In such an event, this oil mixture at the bottom of auger 220 is in good contact with mixed bedding 302a, to allow the oil mixture to be well mixed and absorbed by mixed bedding. At the bottom of auger 220, UV light 310 is provided through an opening in auger 220 to expose mixed bedding 302a to UV light to kill microbes and bacteria (such as e-coli) that may be still present in mixed bedding 302a. In one embodiment, UV light 310 is provided in an array approximately 1 square foot in size and the length of exposure of bedding 302a to the UV light will be at least 1 to 2 seconds to permit killing of bacteria.

As auger 220 is driven by its motor, mixed bedding 302a is moved upward along the length of auger 220 while still being mixed with oil mixtures 304c and 304b. Mixed bedding 302b at the mid-section of auger 220 continues to be propelled upwards toward the upper exit section of auger 220. As mixed bedding 302c leaves auger 220, it is saturated with oil mixture 304. The level of saturation of the mixed bedding with the added oil mixture will depend on factors such as the size of the augers, the speed of auger rotations during processes, the rate of injection of the oil treatment and desired level of saturation. Adjusting any one of these parameters may greatly or minimally impact the throughput and the moisture content of the end product. Once leaving auger 220, mixed bedding 302d is measured into fixed volumes (e.g. between approximately 3 and 12 cubic feet), inserted into bags 226 and then the bags are sealed. The mixed bedding may be compressed when inserted. The weight of the mixed bags may vary (e.g. about between approximately 28 and 38 lbs. depending on content and moisture level). This mixed bedding is the recycled bedding product which is an output of an embodiment.

The throughput of mixed bedding through system 200 is determined by several parameters, including a mixing time in mixing process 102, heating time in drying process 106, and the turn rate of auger 220. Aside from structural changes to the components, speeds and temperatures and even inclines may be controlled through a control panel or manually. In one configuration, the total time of after mixing of the bedding (process 102) to the bagging of the bedding (process 116) is approximately 9 minutes.

In other embodiments, some of the processes in process 100 may be omitted or re-ordered, such as separation process 108, screening process 110 and UV treatment process 112. Typically, adding of the oil mixture process 114 is performed after drying process 106 so that the oil mixture is not chemically altered or ignited by the heat in drying process 106.

The system may be operated by manual controls or through a master control panel with partial or full automation. Automatic responses from various functions of the system may be sent to a master control panel and any necessary production adjustments may be automatically made to improve performance.

Now, further details are provided on exemplary compositions for oil mixture 304 for an embodiment. There are three main components of oil mixture 304, which combine to form an emulsion of: essential oils, a carrier lotion and water. Essential oils are typically plant extract oils. A carrier lotion is a mixture of oils and water where the oils may contain vegetable sourced oils derived from the fatty portion of a plant, usually from the seeds, kernels or nuts. If essential oils are applied to skin undiluted, the essential oils may cause an irritation or reactions in animals. The carrier lotion and water dilute essential oils prior to being added to the mixed bedding material. The carrier lotion also acts as a binding agent for the oil mixture to the mixed bedding.

An exemplary composition of the oil mixture for the mixed bedding, a ratio expressing the relative amount of the essential oil mixture (as a volume) to the carrier lotion (as a volume) to water (as a volume):

Essential oils 250 mL (approx.):Carrier lotion 3750 mL (approx.):Water 16000 mL (approx.) Equation 1

These components of the oil mixture when mixed as noted produces a total volume of approximately 20 L. This 20 L oil mixture is added to approximately 80 bales of mixed bedding, where 1 bale is approximately 3 to 12 cubic feet of compressed bedding weighing approximately 28 to 38 lbs. (12-18 kg). Expressed in terms of volume alone, approximately 250 mL of the oil mixture is added to approximately 85 L (3 cubic feet) of mixed bedding. This concentration level of mixture to bedding has been found to provide an acceptable therapeutic effect for a formulation provided in Tables Al and A2. A lower concentration may be provided with lesser effect. A higher concentration, up to approximately 500 mL per approximately 85 L will strengthen the intensity of the fragrance in the bedding but may not substantially increase the therapeutic effect.

In other embodiments, each component in the ratio in Equation 1 may be adjusted to be higher or lower independently of the other two components by about 25% (by volume), providing a multitude of different ratios.

The following Table Al lists exemplary essential oils that may be used in different combinations and formulations for an oil mixture for an embodiment:

TABLE A1

| Essential Oil | Effects/treatments |
|---|---|
| Black spruce | Fragrance, anti-inflammatory, respiratory issues, antiviral, |
| (Picea mariana) | muscle and joint injuries, fungicidal, antiviral, antibacterial, |
| | insect repellent, parasiticide |
| Canadian balsam | Fragrance, anti-inflammatory, respiratory complaints, |
| (Abies balsamea) | muscle and joint injuries/problems, antifungal, antiviral, |
| | antihistamine, insect repellent, nematicide, antibacterial, |
| | skin irritations and wounds |
| Canadian spruce | Fragrance, wounds, muscle and joint injuries, anti-viral, |
| (Tsuga candensis) | insect repellent, anti-inflammatory, antifungal, |
| | antispasmodic, antibacterial |
| Cassia | Anti-fungal, anti-viral, antispasmodic, anti-inflammatory, |
| (cinnamomum cassia) | joint and muscle issues, mold deterrent, respiratory issues, |
| | insecticide, parasiticide, nematicide. |
| Douglas fir | Fragrance, respiratory problems, muscle and joint injuries, |
| (Pseudotsuga menzieiis) | anti-viral, anti-microbial, countering fungal infections |
| Silver fir | Fragrance, antibacterial, antiviral, antispasmodic, fungicide, |
| (Abies siberica) | respiratory problems, muscle and joint injuries/problems, |
| | insect repellent |
| Peppermint | Fragrance, respiratory issues, muscle and joint problems, |
| (Mentha piperita) | insect repellent, digestive issues |
| Manuka | Relatively expensive, treatment as an anaesthetic, |
| (Leptospermum | antifungal, antimicrobial, anti-inflammatory, antirheumatic, |
| scoparium) | antiseptic, antiviral, bactericide, cytophylactic, |
| | decongestant, expectorant, immuno-stimulant, insecticide, |
| | parasitacide, sedative, tonic |
| Litsea cubeba | Distinctive lemon scent, may irritate mucus membrane and |
| (Litsea cubeba) | skin in certain doses, treatment for respiratory ailments, |
| | muscle and joint issues, insecticidal, digestive upsets, skin |
| | irritations, anti-inflammatory, coughs and phlegm, fungicide, |
| | candidacide |

Table A2 shows additional oils that may also be used in oil mixture formulations. Some effects/features of these oils are listed therein:

TABLE A2

| Essential Oil | Characteristics |
|---|---|
| Himalayan cedar | Relatively expensive; may leave sticky residue |
| (Cedrus deodora) | |
| Frankincense | More expensive |
| (Boswellia | |
| carterii) | |
| Tea tree | Distinctive scent, may irritate mucus membrane and |
| (Melaleuca | may cause excessive sweating in certain doses |
| alternifolia) | |
| Eucalyptus | May be used for certain applications with care taken |
| (Eucalyptus | for its high ketone content, which may cause harm to |
| globulus) | animals or young/small children or pregnant |
| | women. |
| Texas cedarwood | May be used for certain applications with care taken |
| (Juniperus | for its high ketone content, which may cause harm to |
| virgiana) | animals or young/small children or pregnant |
| | women. |

Table B shows exemplary composition percentage ranges, volume amounts and alternative ranges for the composition of essential oils in one formulation of an essential oil mixture for an embodiment:

TABLE B

| | Exemplary | Exemplary Amount | | Acceptable range of |
|---|---|---|---|---|
| | percentage of | of Essential Oils for | | percentages of |
| | Essential Oils | bedding | | Essential Oils |
| Oil | (% by vol.) | embodiment (mL) | | (% by vol.) |
| Black spruce | 09-14% | 24-32 | mL | 0.25-20% |
| Canadian | 12-18% | 36-44 | | 0.25-30% |
| balsam | | | | |
| Douglas fir | 09-11% | 28-32 | | 0.25-15% |
| Silver fir | 25-37% | 76-85 | | 0.25-30% |
| Cassia | 0.5-02% | 01-03 | | 0.025-05% |
| Peppermint | 01-05% | 06-10 | | 0.25-10% |
| Canadian | 22-26% | 60-68 | | 0.25-30% |
| spruce | | | | |
| Litsea | 0.25-02% | 0.5-05 | | 0.20-08% |
| cubeba | | | | |
| Manuka | 0.05-02% | 0.07-02 | | 0.04-04% |
| Total | 100% | 250 | mL | 100% |

The amount of each essential oil listed in Table B express ranges of acceptable values for embodiments. At least one range of values for the exemplary formulation listed in Table B provides essential oil blends that are stable and do not break down in a significant manner through a wide temperature range from about approximately −10° C. to 45° C. The formulations also present combined fragrances that have been found to be pleasing and economical to produce for the properties desired.

The amounts shown in Table B have been determined to be an effective amount for their associated properties. Some oils, when combined, may have complementary properties. For example, having black spruce, Canadian balsam, douglas fir and Canadian spruce as a set of oils in a formulation (which may be considered to be a set of evergreen essential oils) provides an effective treatment for joint mobility and range issues, respiratory issues by assisting in opening up the air passageways for the horses that inhale the oil mixture. In another formulation, black spruce may be provided with none or one or more of Canadian balsam, douglas fir and Canadian spruce as black spruce has been found to be a dominant oil for treatments. Other formulations based on evergreen oils from Canadian balsam, douglas fir, and Canadian spruce have been determined to be effective for treating respiratory issues and providing antiviral and antifungal properties. Cassia has been found to be effective as an anti-mold treatment, an analgesic, anti-inflammatory, anti-viral, anti-fungal in relatively low concentrations and can be added to formulations where these treatments are desired. At higher concentrations it may be an irritant to skin and mucus membranes. The column showing the mL content of exemplary oils totaling 250 mL shows volumes of oils for a specific oil blend used in an oil mixture for the mixed bedding described below. The columns showing the percentages may be used in other applications (e.g. sprays, lotions, creams) described below.

In other embodiments, selected oils may be eliminated or reduced from formulations described above. For example, some essential oils may have undesirable effects for a target formulation in certain doses and so they may not typically be used that formulation—note that the blends in Table B include litsea cubeba and manuka, which may be excluded in certain formulations. Essential oils that contain ketones may not be typically suitable as the ketones may make these oil mixture hepato toxic, abortifacient, neurotoxic, and may cause mucus membrane irritations. However, if the dosages are properly moderated, they may still be used in a given formulation. Exemplary essential oils that have a higher ketone component include western red cedar (thuja plicata), atlas cedar (cedrus atlantica), and scotch pine (pinus sylvestris).

For the carrier lotion, Table C shows exemplary composition percentage ranges, volume amounts and alternative ranges for the composition of carrier oils per the oil itself in one formulation of a carrier lotion for an embodiment:

TABLE C

| | Exemplary | | | | | |
|---|---|---|---|---|---|---|
| | percentage | | | | | |
| | of carrier | | | | | |
| | oils | Exemplary | | Exemplary | | |
| Ingredient | (% by vol.) | Amount (mL) | | Amount (g) | | Notes |
| Cetyl alcohol | 15-20% | 85-105 | mL | 40-50 | g | May be derived from |
| | | | | | | coconut oil |
| Stearic acid | 40-60% | 225-330 | mL | 140-165 | g | Fatty acid derived from |
| | | | | | | coconut oil |
| Caprylic acid | 10-20% | 35-75 | mL | 25-55 | g | Fatty acid, also known |
| | | | | | | as fractionated coconut |
| | | | | | | oil, stable, fungicide |
| Polysorbate 20 | 1-5% | 3-15 | mL | 2-11 | g | Derived from lauric |
| | | | | | | acid in coconut oil |
| Borax | 3-7% | 9-25 | mL | 8-18 | g | Mineral salt, softens |
| (sodium borate, | | | | | | water, makes water |
| sodium | | | | | | “wetter”, fungicide |
| tetraborate, or | | | | | | |
| disodium | | | | | | |
| tetraborate) | | | | | | |
| Total | 100% | Approx. 495 | mL | Approx. 266 | g | |

The column showing the mL content of exemplary ingredients totaling 495 mL shows volumes of ingredients for a carrier lotion used with an oil mixture for the mixed bedding described below. The carrier lotion may have the consistency resembling toothpaste or a body lotion.

Component oils in the carrier lotion may also have treatment properties. With the treatment properties of the essential oils and the carrier lotion, adding specific additional antimycotic or antibacterial agents may not be necessary, but can be added if desired. For example, beeswax may be used as an alternative or additive in lieu of the stearic acid in the carrier lotion, but its cost may not make the product economical.

Another aspect of an embodiment provides a blend of essential oils and water for direct application to an animal as a spray. This blend is not mixed with mixed bedding. The spray provides therapeutic effect to protect against viruses, parasites and bacteria present in other animals, insects and water. It may be applied by lightly misting it over the entire animal, with care taken to not apply the spray in the eyes of the animal.

The blend of essential oils provided in Table B provide compositions that may be mixed with an amount of carrier lotion and water (different from Equation 1) and applied as a spray through a spray bottle or other suitable aspirator, such as an automated spray dispenser. Other essential oil blends may be provided in a spray. For example, Table D provides composition percentage ranges, volume amounts and alternative ranges for a composition of essential oils in another formulation of an essential oil mixture for an embodiment, where the mixture destined for a spray formulation applied directly to animals' bodies as a spray.

TABLE D

| | Exemplary | Alternative |
|---|---|---|
| | Formulation Amount | acceptable range in |
| Oil | in Mixture (% by vol.) | Mixture (% by vol.) |
| Litsea cubeba | 0.25-6% | 0.5-40% |
| Manuka | 0.05-4% | 0.07-20% |
| Peppermint | 1-5% | 0.25-10% |
| Cassia | 0.5-2% | 1-3% |

For variations for spray compositions shown in Table D, one or more of any combination of the oils may be provided in the exemplary ranges shown. Additional oils may be added.

Exemplary compositions of sprays comprise a combination of an essential oil blend to carrier lotion (exemplary compositions described earlier) to water.

Another aspect of an embodiment provides a blend of essential oils, carrier lotion and water for direct application to an animal as a cream. The cream may be effective to counter inflammations, arthritis/joint injuries, sprains, strains, fungal and yeast infections. The cream is intended to be applied directly to the area affected of the animal.

For a cream composition, the essential oil and carrier lotion compositions can be based on any of the respective formulations described earlier. An exemplary composition of the cream as a ratio expressing the relative amount of the essential oil mixture (as a volume) to the carrier lotion (as a volume) is:

Essential oils 8.5 mL (approx.):Carrier lotion 410 mL (approx.) Equation 2

Some water may be added to the resulting cream to increase its water content (making it more spreadable).

Another aspect of an embodiment provides a blend of essential oils as a concentrate, which is applied directly to an animal for treatment of issues such as bites, hoof rot, scrapes, cuts, sores and to stimulate the normal healing response to other irritations occurring on the animal. An exemplary composition of the concentrate as a ratio expressing the relative amount of the essential oil mixture (as a volume) to the carrier lotion (as a volume) to water for a 4 oz. (US) container is:

Essential oils 6 mL (approx.):Carrier lotion 6 mL (approx.):Water (approx.) 108 mL 3A

A variant embodiment for the concentrate for a spray has a blend of essential oils and water as a ratio as:

Essential oils 1 mL (approx.):Water (approx.) 99 mL tm Equation 3B

Another embodiment provides another spray formulation as a concentrate, which is applied directly to an animal onto the affected area ensuring that it is kept out the eyes. Different formulations of the spray are provided for treatment and ease of respiratory and digestive issues, including flatulence, colic, colds, catarrh and allergies. An exemplary composition of this other concentrate as a ratio expressing the relative amount of the essential oil mixture (as a volume) to the carrier lotion (as a volume) to water for a 20 oz. (US) container is:

Essential oils 12 mL (approx.):Carrier lotion 12 mL (approx.):Water (approx.) 576 mL Equation 3C

Another embodiment provides another spray formulation as a concentrate, which is applied directly to an animal or human to protect against airborne carriers of bacteria and viruses, flies, mosquito and other critters. This formulation and its variants contain cassia and peppermint extracts as provided in Table D. Exemplary compositions of this other concentrate as ratios expressing the relative amount of the essential oil mixture (as a volume) to the carrier lotion (as a volume) to water may follow the ratios shown in Equation 3A (for a 4 oz. US portion or) and Equation 3C (for a 20 oz. US portion).

It will be appreciated that the processes, systems and formulations for embodiments described herein relate to treating used bedding material. Other embodiments may process other materials, such as woodchips, grains, minerals, seeds, recycled wood fiber, sawdust, wood shavings, wood pellets, straw, chopped straw, shredded paper, hemp/flax, peat moss, etc.

As used herein, the wording “and/or” is intended to represent an inclusive-or. That is, “X and/or Y” is intended to mean X or Y or both.

In this disclosure, where a threshold or measured value is provided as an approximate value (for example, when the threshold is qualified with the word “about”), a range of values will be understood to be valid for that value. For example, for a composition value or threshold stated as an approximate value, a range of about 25% larger and 25% smaller than the stated value may be used. Thresholds, values, measurements and dimensions of features are illustrative of embodiments and are not limiting unless noted.

Although a preferred embodiment has been disclosed for purposes of illustration, it should be understood that various changes and modifications and substitutions could be made in the preferred embodiment without departing from the spirit of the invention as defined by the claims which follow.

1. A method of processing animal bedding material, comprising:
mixing used bedding with unused bedding to produce mixed bedding;
deodorizing the mixed bedding;
drying the mixed bedding to have a moisture content of between approximately 7 and 14%;
removing lighter fibers from the mixed bedding;
adding an oil treatment to the mixed bedding to have a moisture content of between approximately 7 and 14% in the mixed bedding; and
bagging the mixed bedding while the mixed bedding is still moist from the oil treatment.

2. The method of claim 1, further comprising:
deodorizing the mixed bedding with bicarbonate soda after the used bedding is mixed with the unused bedding.

3. The method of claim 1, wherein the oil treatment is added through an auger on an incline that mixes the mixed bedding with the oil treatment.

4. The method of claim 3, wherein the oil treatment comprises a formulation of essential oils comprising:
between approximately 9 and 14% of black spruce essential oil.

5. The method of claim 4, wherein the formulation of essential oils further comprises:
between approximately 12 and 18% of Canadian balsam essential oil;
between approximately 9 and 11% of douglas fir essential oil; and
between approximately 22 and 26% of Canadian spruce essential oil.

6. The method of claim 5, wherein the formulation of essential oils further comprises:
between approximately 0.5 and 2% of cassia.

7. The method of claim 6, wherein the formulation of essential oils further comprises:
between approximately 25 and 37% of silver fir essential oil;
between approximately 0.25 and 6% of litsea cubeba essential oil;
between approximately 1 and 5% of peppermint essential oil; and
between approximately 0.5 and 2% of cassia essential oil.

8. The method of claim 4, wherein the oil treatment further comprises a carrier lotion that comprises:
between approximately 15 and 20% of cetyl alcohol;
between approximately 40 and 60% of stearic acid;
between approximately 10 and 20% of caprylic acid;
between approximately 1 and 5% of polysorbate 20; and
between approximately 3 and 7% of Borax.

9. The method of claim 8, wherein a composition of the oil treatment comprises a ratio of the essential oils to the carrier lotion to water as being approximately:
essential oils 1 : carrier lotion 0.933 : water 0.0765.

10. The method of claim 9, wherein approximately 18.9 L of the oil treatment is mixed with approximately 80 bales of the mixed bedding.

11. An oil treatment for treating animal bedding material, comprising a formulation of essential oils comprising:
between approximately 9 and 14% of black spruce essential oil; and
between approximately 0.5 and 2% of cassia.

12. The oil treatment as claimed in claim 11, wherein the formulation of essential oils further comprises:
between approximately 12 and 18% of Canadian balsam essential oil;
between approximately 9 and 11% of douglas fir essential oil; and
between approximately 22 and 26% of Canadian spruce essential oil.

13. The oil treatment as claimed in claim 12, further comprising a carrier lotion that comprises:
between approximately 15 and 20% of cetyl alcohol;
between approximately 40 and 60% of stearic acid;
between approximately 10 and 20% of caprylic acid;
between approximately 1 and 5% of polysorbate 20; and
between approximately 3 and 7% of Borax.

14. The oil treatment as claimed in claim 13, wherein the oil treatment comprises a ratio of the essential oils to the carrier lotion to water as approximately:
essential oils 250: carrier lotion 3750 : water 16000.

14. The oil treatment as claimed in claim 14, wherein approximately 18.9 L of the oil treatment is mixed with approximately 240 cubic feet of the mixed bedding.

16. An oil treatment for application to an animal, comprising a formulation of essential oils comprising:
between approximately 0.25 and 6% of manuka essential oil; and
between approximately 0.05 and 4% of litsea cubeba essential oil.

17. The oil treatment as claimed in claim 16, wherein the formulation of essential oils further comprises:
between approximately 9 and 14% of black spruce essential oil.
between approximately 12 and 18% of Canadian balsam essential oil;
between approximately 9 and 11% of douglas fir essential oil; and
between approximately 22 and 26% of Canadian spruce essential oil.

18. The oil treatment as claimed in claim 17, further comprising a carrier lotion that comprises:
between approximately 15 and 20% of cetyl alcohol;
between approximately 40 and 60% of stearic acid;
between approximately 10 and 20% of caprylic acid;
between approximately 1 and 5% of polysorbate 20; and
between approximately 3 and 7% of Borax.

19. The oil treatment as claimed in claim 18, wherein the oil treatment comprises a ratio of the essential oils to the carrier lotion to water as approximately:
essential oils 250: carrier lotion 3750 : water 16000.
